(19) United States
(12) Patent Application Publication
(10) Pub. No.: US 2018/0153601 A1
(43) Pub. Date: Jun. 7, 2018

(54) SURGICAL PROCEDURE

(71) Applicants: A. Jamie Riley, St. Louis, MO (US); Gary J. Schmidt, St. Louis, MO (US)

(72) Inventors: A. Jamie Riley, St. Louis, MO (US); Gary J. Schmidt, St. Louis, MO (US)

(21) Appl. No.: 15/833,379

(22) Filed: Dec. 6, 2017

Related U.S. Application Data
(60) Provisional application No. 62/430,665, filed on Dec. 6, 2016.

(51) Int. Cl. A61B 17/86; A61B 17/82; A61B 17/84; A61B 17/04

(52) U.S. Cl. CPC A61B 17/8685; A61B 17/82; A61B 2017/681; A61B 17/0401; A61B 17/842

(57) ABSTRACT

A surgical procedure for inducing continuous compressive force across two components includes inserting a braided superelastic wire through a first end of a channel that from a first component to a second component. The braided superelastic wire includes an unstretched length in a contracted state. The procedure also includes anchoring the braided superelastic wire at a second end of the channel opposite the first end and applying a predetermined amount of axial tension to the braided superelastic wire adjacent the second end of the channel. The axial tension stretches the braided superelastic wire to a stretched state having a stretched length that is longer than the unstretched length. The procedure also includes securing at least one free end of the braided superelastic wire adjacent the second end of the channel to maintain the predetermined amount of axial tension along the braided superelastic wire across the first component and the second component. The braided superelastic wire is configured to be biased toward the contracted state when in the stretched state such that a compressive spring force is applied by the wire while in the stretched state.

### CROSS REFERENCE TO RELATED APPLICATION

This application is a non-provisional application and claims priority to U.S. Provisional Patent Application Ser. No. 62/430,665 filed Dec. 6, 2016, for “SURGICAL PROCEDURE”, which is hereby incorporated by reference in its entirety.

### BACKGROUND

The present invention relates generally to surgical procedures and, more particularly, to an improved surgical system and method for inducing constant compressive force across two components, for example, bones.

In severe trauma to the ankle with a rotatory moment, the contact area between the tibia and fibula directly above the ankle (the syndesmosis) can become disrupted. Disruption of the syndesmosis is an indication for surgical correction. Failure to repair the disruption can lead to permanent arthritic damage to the ankle joint. Repair has typically been performed with screws between disrupted components of the syndesmosis, but research shows that using screws may lead to chronic pain and/or malreduction, which may only be evident on a CT scan. Repair has also been performed using thread-based suture methods, which have been shown to be more reliable than screw-based repair. However, suture methods are limited by the amount of compression the thread can provide and by the strength of the thread itself, which can break if excessive force is applied thereto. Such problems may occur in other surgical procedures involving the compression of two components (i.e., bones) together.

Therefore, it would be advantageous to provide a surgical method and system that can provide the reliability of suture-based repair methods with a constant, predetermined amount of force through the lifetime of the suture, wherein such systems and methods can be adapted to a variety of joints.

### BRIEF SUMMARY

In one aspect, a surgical procedure for inducing continuous compressive force across two components is provided. The surgical procedure includes inserting a braided superelastic wire through a first end of a channel that from a first component to a second component. The braided superelastic wire includes an unstretched length in a contracted state. The procedure also includes anchoring the braided superelastic wire at a second end of the channel opposite the first end and applying a predetermined amount of axial tension to the braided superelastic wire adjacent the second end of the channel. The axial tension stretches the braided superelastic wire to a stretched state having a stretched length that is longer than the unstretched length. The procedure also includes securing at least one free end of the braided superelastic wire adjacent the second end of the channel to maintain the predetermined amount of axial tension along the braided superelastic wire across the first component and the second component. The braided superelastic wire is configured to be biased toward the contracted state when in the stretched state such that a compressive spring force is applied by the wire while in the stretched state.

In another aspect, a surgical system is provided. The surgical system includes a braided superelastic wire configured to induce a compressive force across a first component and a second component through a channel. The channel includes a first end through at least a portion of the first component and a second end through at least a portion of the second component. The braided superelastic wire includes an unstretched length and is further configured to stretch to a stretched length, which is longer than the unstretched length, when an axial force is applied thereto. The braided superelastic wire is further configured to contract back to its unstretched length when the axial force is removed. The system also includes an anchor configured to anchor the braided superelastic wire adjacent the second end of the channel and to prevent the braided superelastic wire from being displaced through the channel upon application of the predetermined amount of axial force to the braided superelastic wire.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 illustrates a first perspective view of a braided superelastic wire in an unextended state for use in surgical systems in accordance with the present disclosure.

FIG. 2 illustrates a second perspective view of the braided superelastic wire shown in FIG. 1 with an axial force exerted thereon.

FIG. 3a illustrates a top view of a braided superelastic ribbon in an unextended state for use in surgical systems and procedures in accordance with the present disclosure.

FIG. 3b illustrates a top view of the braided superelastic ribbon with an axial force exerted thereon.

FIG. 4a illustrates a side view of the braided superelastic ribbon in an unextended state.

FIG. 4b illustrates a side view of the braided superelastic ribbon with the axial force exerted thereon.

FIG. 5 is a first view of a first region of a patient's body on which a first surgical procedure is to be performed.

FIG. 6 is a second view of the region shown in FIG. 5 with a cannulated drill therethrough in accordance with one aspect of the first surgical procedure.

FIG. 7 is a third view of the region shown in FIGS. 5 and 6 with the braided superelastic wire or ribbon shown in FIGS. 1-4 disposed therethrough to induce a compressive force across the region.

FIG. 8 is a first view of a second region of a patient's body on which a second surgical procedure is performed, with a guidewire therethrough in accordance with one aspect of the second surgical procedure

FIG. 9 is a second view of the second region shown in FIG. 8 with the braided superelastic wire or ribbon shown in FIGS. 1-4 disposed therethrough to induce a compressive force across the region.

FIG. 10 is a perspective view of a third region of a patient's body on which a third surgical procedure is performed by wrapping the braided superelastic wire or ribbon shown in FIGS. 1-4 around the third region.

FIG. 11 is a front view of a fourth region of a patient's body on which the third surgical procedure is performed by wrapping the braided superelastic wire or ribbon shown in FIGS. 1-4 around the fourth region.

FIG. 12 is a first view of a fifth region of a patient's body on which a fourth surgical procedure is to be performed.

FIG. 13 is a second view of the fifth region of a patient's body on which the fourth surgical procedure is to be performed.

### DETAILED DESCRIPTION

In the following detailed description numerous specific details are set forth in order to provide a thorough understanding of the disclosure. However, it will be understood by those skilled in the art that the present disclosure may be practiced without these specific details. For example, the disclosure is not limited in scope to the particular type of industry application depicted in the figures. In other instances, well-known methods, procedures, and components have not been described in detail so as not to obscure the present disclosure.

The embodiments described herein include clinical utilization of a braided superelastic wire or ribbon formed from nitinol and created in a fashion such that a woven pattern will create elasticity within the strand, thus preserving the strength qualities of the nitinol. The braided superelastic wire or ribbon will be utilized in orthopedic applications utilizing an anchor device to the bone, a securing “button device” as well as a compressive device. This procedure is utilized in repair of bone-to-bone, tendon-to-bone, or ligament-to-bone. The nitinol braided superelastic wire or ribbon provides a low profile material with a small footprint that the completely biocompatible and also provides a constant measured pressure with strength that is well beyond physiologic requirements.

The method described herein includes an anchor device placed within cancellous bone. This anchor device is used with a superelastic dynamic woven attachment mechanism in the form of a “wire” or “ribbon”. Typically a drill hole is formed within the bone itself and the anchor is turned into the bone similar to the method used to place a screw. The anchor is used to engage either the cancellous bone or the far cortex of the bone involved.

The braided superelastic attachment mechanism extends into or through one bone in the case when one is trying to attach a tendon or ligament to the bone or into or through two or more bones if one is trying to attach a bone to another bone in the case of repair of a fracture, or trying to create a fusion between two bones. With release of the anchor within the bone, completed by removal of a handle apparatus, the “wire” or “ribbon” attachment mechanism is anchored deeply within the bone and from the selected end. In the case where the attachment mechanism passes between two or more bones or bone fragments, a button device is placed and compressed at this point and a tension device would measure the amount of tension within the system. In the case where one is securing a ligament or tendon to the bone, the “rope” or “ribbon” itself may be passed through the tendon securing it to the bone, or a similar button may be used.

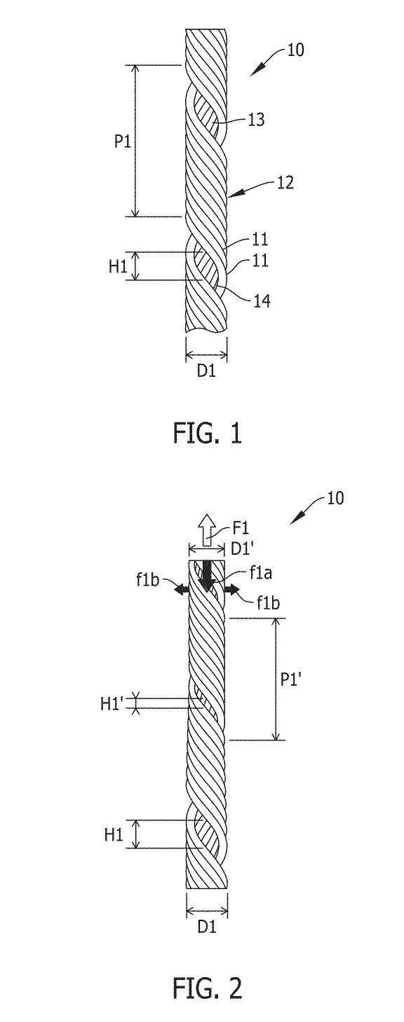

FIG. 1 illustrates a first perspective view of a braided superelastic wire 10 in an unextended state for use in the surgical procedures and systems described further herein, and FIG. 2 illustrates a second perspective view of wire 10 with an axial tension force F1 exerted thereon. In the exemplary embodiment, wire 10 is fabricated from a braided superelastic material. Wire 10 includes a generally spiral base body 12 including a plurality of individual wire strands 11. Strands 11 are wound or woven spirally at a pitch P1 to form base body 12 having a diameter D1. Gaps 14 are formed in base body 12 along an axial direction, adjacent a void or cavity 13 of wire 10 that is circumscribed by strands 11. Gaps 14 have a height H1 taken between adjacent strands 11 on either side of gap 14. Strands 11 are fabricated from a material having a shape memory and as well as a large elastic force, and a number of other particular properties including tensile strength, stretching properties, flexibility, and compressibility to form wire 10 having desired properties, as described further herein. Accordingly, strands 11 may be fabricated from nitinol. In a specific embodiment, the nitinol wire comprises stainless steel, titanium alloys, Co—Cr alloys, other non-ferrous based metals or metal alloys, and/or a combination thereof. More particularly, in one embodiment, strands 11 are fabricated from a component ratio as follows: Nitrogen, 0.05% or less; carbon, 0.08% or less; hydrogen, 0.012% or less; iron, 0.25% or less; oxygen, 0.13% or less; aluminum, 5.5% to 6.5%, vanadium, 3.5% to 4.5%, and the balance titanium. Wire 10 has a number of unique characteristics resulting from its multi-threaded metal structure. Notably, wire 10 exhibits elastic properties in that wire 10 can stretch or extend in an axial direction.

As shown in FIG. 2, when axial force F1 is exerted on wire 10, the “spiral” of base body 12 becomes “steeper.” More particularly, pitch P1 of strands 11 increases to P1′, which causes gaps 14 to narrow to a height of H1′, and the diameter of wire 10 decreases to D1′. This adjustment generates a compressive force f1a that extends axially opposite force F1, and a radial expansion force f1b that urges strands 11 to return to their original position (shown in FIG. 1). Once force F1 is removed, forces f1a and f1b act to return wire 10 to its original, contracted, or unstretched state. In other words, once stretched, wire 10 naturally exerts forces to return wire 10 to its initial, unstretched state once Force F1 is removed. If maintained in a stretched or extended configuration, i.e., if force F1 is maintained in the axial direction, wire 10 will continuously exert compressive or contractive force f1a. Unlike other elastic materials, which may lose elasticity or contractibility after a certain amount of time in a stretched state, wire 10 maintains its elasticity substantially throughout its usable lifetime. In operation, axial compressive force f1a is proportional to axial tension force F1 such that as force F1 increases, so does force f1a. When a constant axial tension force F1 is applied to wire 10, wire 10 reacts by applying a constant axial compressive force f1a as long as axial tension force F1 is applied.

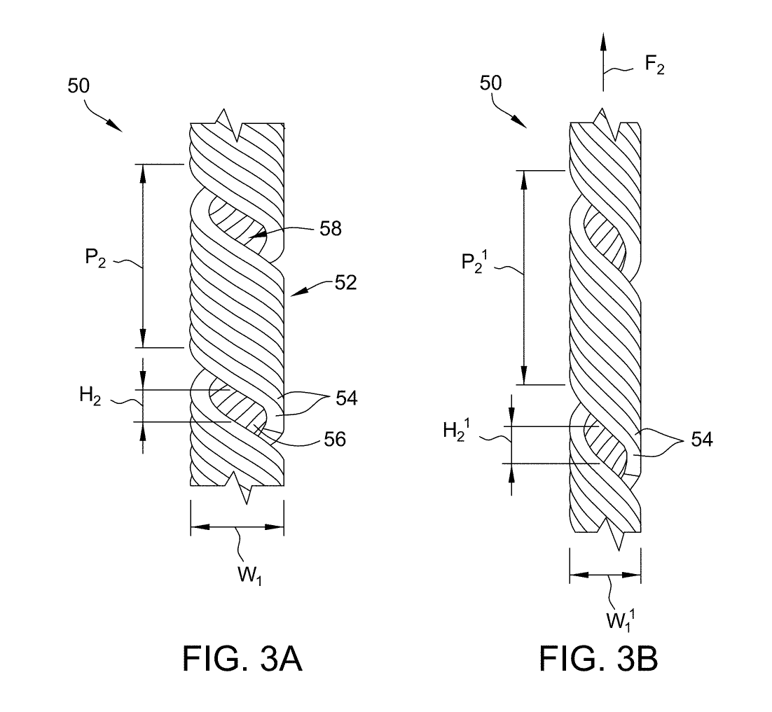

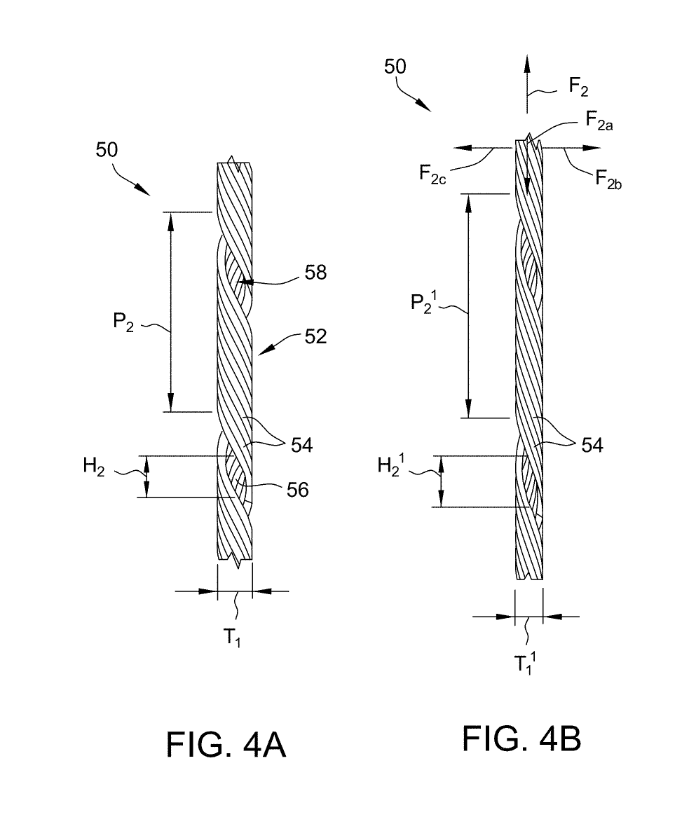

FIG. 3a illustrates a top view of a braided superelastic ribbon 50 in an unextended state for use in surgical systems and procedures in accordance with the present disclosure. FIG. 3b illustrates a top view of braided superelastic ribbon 50 with an axial force F2 exerted thereon. FIG. 4a illustrates a side view of braided superelastic ribbon 50 in an unextended state. FIG. 4b illustrates a side view of braided superelastic ribbon 50 with axial force F2 exerted thereon. In the exemplary embodiment, similar to wire 10 as described above, ribbon 50 is fabricated from a braided superelastic material. As shown in FIGS. 3a and 3b, ribbon 50 includes a body portion 52 including a plurality of individual wire strands 54. Strands 54 are wound or woven spirally at a pitch P2 to form a base portion 52 having a width W1 and a thickness T1. In the exemplary embodiment, width W1 includes a range of approximately 1.0 millimeters (mm) to approximately 10.0 mm. More specifically, width W1 includes a range of approximately 2.0 mm to approximately 5.0 mm. More specifically, width W1 includes a range of approximately 3.0 mm to approximately 4.0 mm. Even more specifically, width W1 includes a range of approximately 3.2 mm to approximately 3.5 mm. Similarly, in the exemplary embodiment, thickness T1 includes a range of approximately 0.3 mm to approximately 3.0 mm. More specifically, thickness T1 includes a range of approximately 0.6 mm to approximately 1.6 mm. More specifically, thickness T1 includes a range of approximately 1.0 mm to approximately 1.5 mm. Generally, ribbon 50 includes a ratio of width W1 to thickness T1 within a range of approximately 2:1 to approximately 5:1, and, more specifically a ratio of approximately 3:1.

Body portion 52 has a substantially rectangular cross-section that defines a cavity 58. Gaps 56 are formed in body portion 52 along an axial direction, adjacent a void or cavity 58 of w ribbon 50 that is circumscribed by strands 54. Gaps 56 have a height H2 taken between adjacent strands 54 on either side of gap 56. Strands 54 are fabricated from a material having a shape memory and as well as a large elastic force, and a number of other particular properties including tensile strength, stretching properties, flexibility, and compressibility to form ribbon 50 having desired properties, as described further herein. Accordingly, strands 54 may be fabricated from nitinol. In a specific embodiment, the nitinol wire comprises stainless steel, titanium alloys, Co—Cr alloys, other non-ferrous based metals or metal alloys, and/or a combination thereof. More particularly, in one embodiment, strands 54 are fabricated from a component ratio as follows: Nitrogen, 0.05% or less; carbon, 0.08% or less; hydrogen, 0.012% or less; iron, 0.25% or less; oxygen, 0.13% or less; aluminum, 5.5% to 6.5%, vanadium, 3.5% to 4.5%, and the balance titanium. Ribbon 50 has a number of unique characteristics resulting from its multi-threaded metal structure. Notably, ribbon 50 exhibits elastic properties in that wire 10 can stretch or extend in an axial direction.

As shown in FIGS. 3b and 4b, when axial force F2 is exerted on ribbon 50, the “spiral” of body portion 52 becomes “steeper.” More particularly, pitch P2 of strands 54 increases to P2′, which causes gaps 56 to narrow to a height of H2′, the width W1 of ribbon 50 decreases to W1′, and the thickness T1 of ribbon 50 decreases to T1′. This adjustment generates a compressive force f2a that extends axially opposite force F2, and a radial expansion force f2b that urges strands 54 to return to their original position (shown in FIGS. 3a and 4a). Once force F2 is removed, forces f2a and f2b act to return ribbon 50 to its original, contracted, or unstretched state. In other words, once stretched, ribbon 50 naturally exerts forces to return ribbon 50 to its initial, unstretched state once Force F2 is removed. If maintained in a stretched or extended configuration, i.e., if force F2 is maintained in the axial direction, ribbon 50 will continuously exert compressive or contractive force f2a. Unlike other elastic materials, which may lose elasticity or contractibility after a certain amount of time in a stretched state, ribbon 50 maintains its elasticity substantially throughout its usable lifetime. In operation, axial compressive force f2a is proportional to axial tension force F2 such that as force F2 increases, so does force f2a. When a constant axial tension force F2 is applied to ribbon 50, ribbon 50 reacts by applying a constant axial compressive force f2a as long as axial tension force F2 is applied.

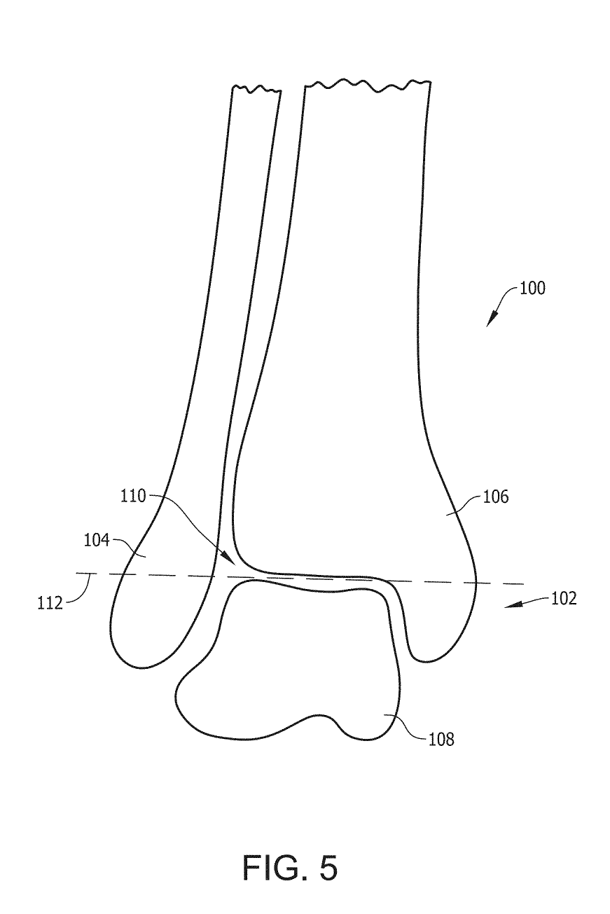

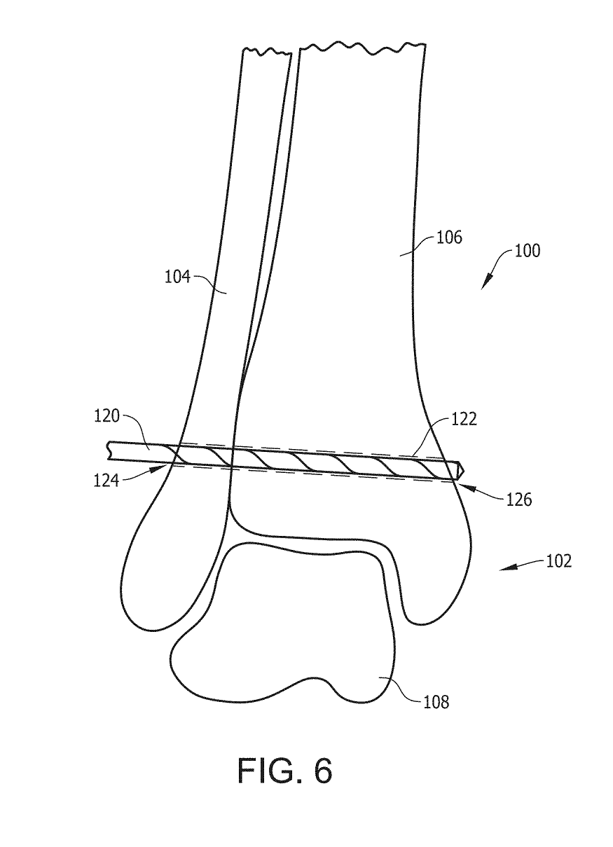

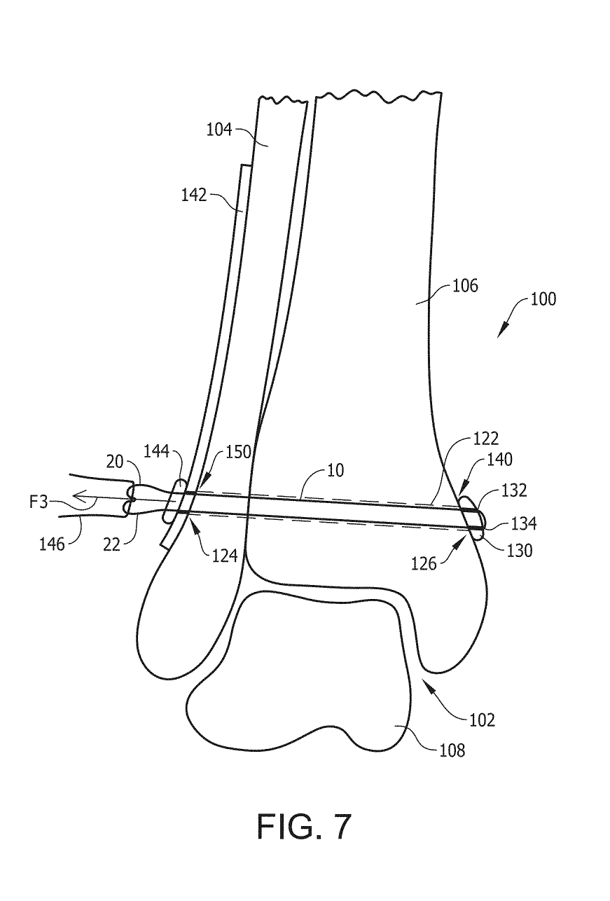

FIGS. 5-7 illustrate a first surgical procedure implemented using wire 10 (shown in FIGS. 1 and 2). More particularly, FIG. 5 is a first view of a region 100 of a patient's body on which the first surgical procedure is to be performed, FIG. 6 is a second view of region 100 with a cannulated drill 120 therethrough in accordance with one aspect of the first surgical procedure, and FIG. 7 is a third view of region 100 with wire 10 disposed therethrough to induce a compressive force across region 100. In the illustrated embodiment, region 100 of FIGS. 5-7 depicts a rear view of a human left ankle joint 102, more particularly a fibula 104, tibia 106, and talus 108, as well as a syndesmosis region 110, which may be torn or otherwise injured. In order to repair the injured syndesmosis region 110, a surgical procedure to draw and compress fibula 104 and tibia 106 together is performed. To reduce or eliminate the above-described issues with traditional surgical procedures, a syndesmotic repair procedure implemented using wire 10 to induce a continuous compressive force across fibula 104 and tibia 106 is herein described. Directional terms may be understood as follows: with respect to the view of FIGS. 5-7, “superior” indicates generally “upwards” in the vertical or longitudinal direction, “inferior” indicates generally “downwards” in the vertical or longitudinal direction, “medial” indicates generally “right” in the horizontal direction (i.e., extending toward the middle of the patient), and “lateral” indicates generally “left” in the horizontal direction (i.e., extending away from the middle of the patient.

Although not shown in FIGS. 5-7, a number of preparatory steps are performed to access region 100. For example, the skin and subcutaneous tissues are incised through a lateral approach to fibula 104 proximate to the level of the superior ankle joint. Care should be taken to avoid the peroneal tendons and sural nerve. These steps may be performed for surgical procedures other than a syndesmotic repair, for example, a repair procedure of an inferior fracture of fibula 104. A clamp (not shown) may be used to compress fibula 104 and tibia 106 together, into a compressed configuration as shown in FIGS. 6 and 7. Under fluoroscopic guidance, a k-wire or guidewire (not shown) is then placed across ankle joint 102, through fibula 104 into tibia 106 parallel to a superior joint line 112 of ankle 102. Because of the general “dome” shape of ankle 102, it is recommended that the guidewire be positioned within approximately one centimeter of joint line 112, to avoid intra-articular penetration.

Once the position of the guidewire is shown to be correct (e.g., verified fluoroscopically), a cannulated drill 120 is passed over the guide wire completely across fibula 104 and tibia 106 to form a channel 122 through ankle 102, as shown in FIG. 6. Channel 122 has a lateral end 124 and a medial end 126. The guidewire and cannulated drill 120 may then be removed. A needle (not shown) is guided through channel 122 from lateral end 124 to medial end 126. The needle has wire 10 and a button 130 coupled thereto. Button includes a first button aperture 132 and a second button aperture 134, through which wire 10 or ribbon 50 is pre-threaded prior to being passed through channel 122. In one embodiment, the needle is passed through skin on the medial side of ankle 102, and draws button 130 through medial end 126 to couple flush against the medial side of tibia 106. In the illustrated embodiment, button 130 is positioned flush on a lateral second metatarsal base 140. It should be understood that button 130 may have any configuration, shape, and/or size such that button 130 functions as described herein. Additionally or alternatively, button 130 may include any suitable anchoring mechanism that functions as described herein.

At this point, wire 10 is anchored on the medial side of tibia 106, with two free ends 20 and 22 loose or free on the lateral side of fibula 104. In some embodiments, ends 20 and 22 of wire 10 may be drawn through one or more apertures (not shown) in a plate 142 positioned against the lateral side of fibula 104, to distribute pressure exerted about lateral end 124 of channel 122. Although the procedures described herein include using wire 10, it is contemplated that ribbon 50 (shown in FIGS. 3a, 3b, 4a, and 4b) may be used instead of wire 10. As such, the procedures are described below as using wire 10 for convenience only and are not meant to be limiting to only the use of wire 10.

In the example embodiment, the position of wire 10 and/or button 130 in channel 122 is verified (e.g., fluoroscopically), at which point a force F3 is applied axially to wire 10 (i.e., parallel to channel 122). A predetermined amount of force is applied to wire 10, to compress fibula 104 and tibia 106 together across channel 122. Applying force F3 causes button 130 to flatten against tibia 106, preventing wire 10 from pulling out of ankle 102. The predetermined amount of force may be determined based on, for example, an amount of compression required between fibula 104 and tibia 106 to appropriately facilitate healing of syndesmotic region 110 or either of fibula 104 and tibia 106. In the illustrated embodiment, force F3 is applied to ankle 102 and is measured using a tensioning device 146. Tensioning device 146 is any device suitable to measure applied force, for example, a spring scale or a force gauge. Using tensioning device 146, force F3 is changed, i.e. increased, until the measured amount of force F3 is substantially equal to a predetermined desired amount of force known to facilitate healing of region 100.

Once the predetermined desired amount of force F3 is applied to wire 10 to appropriately compress fibula 104 and tibia 106 together, free ends 20 and 22 are further coupled to an anchor 144. For example, free ends 20 and 22 may be passed through apertures (not shown) of anchor 144 or may be drawn about threads (not shown) of anchor 144. Anchor 144 may be any anchoring component suitable to anchor free ends 20 and 22, such as but not limited to a second button or a screw. In the illustrated embodiment, plate 142 is positioned to sit flush proximal the lateral malleolus 150 and anchor 144 is coupled to plate 142 such that plate 142 is positioned between anchor 144 and lateral malleolus 150. Alternatively, plate 142 is omitted and anchor 144 is positioned to sit flush proximal the lateral malleolus 150. Ends 20 and 22 of wire 10 are anchored, crimped, and/or cut to a suitable length (e.g., approximately flush to plate 142, if used, or fibula 104). X-ray may be used at this point to verify the position of wire 10. A second wire 10 may be used if necessary following the same procedure at a proximal or distal location.

Accordingly, by performing the above-described surgical procedure, the fibula 104 and tibia 106 are continuously compressed together across channel 122 by wire 10, which continuously exerts a constant compressive force opposite F3. More specifically, as tension force F3 is applied to wire 10 by tensioning device 146, wire 10 stretches to a length longer than its at rest length when no tension is applied. As described above, when wire 10 is secured in such a stretched state, the constant compressive force F3 is maintained because wire 10 includes properties that cause wire 10 to bias towards its contracted or unstretched state when wire 10 is in its stretched state such that a compressive spring force is applied by wire 10 while in the stretched state. As such, when wire 10 is subjected to the constant predetermined desired tension force F3 and then anchored using anchor 144, wire 10 exerts a corresponding constant compressive force f1a (shown in FIG. 2) that continuously compresses fibula 104 and tibia 106 together for as long as wire 10 remains in place.

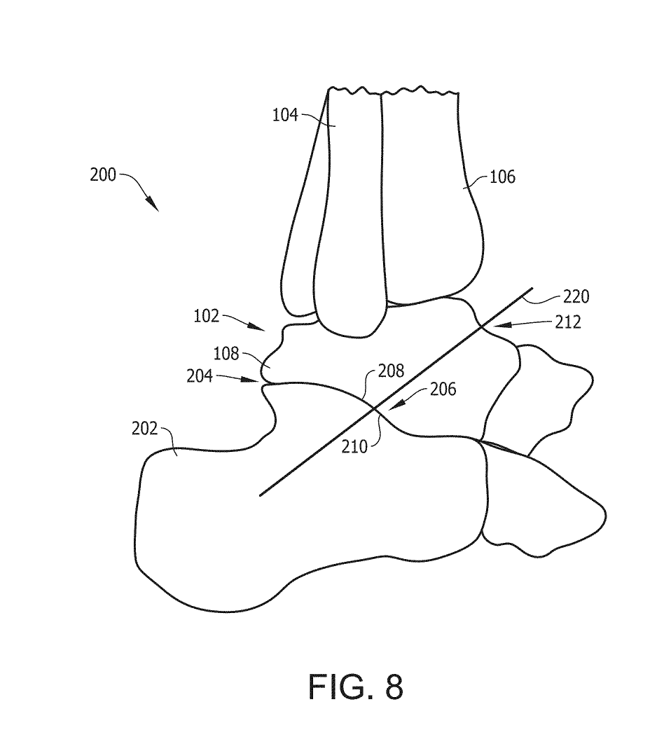

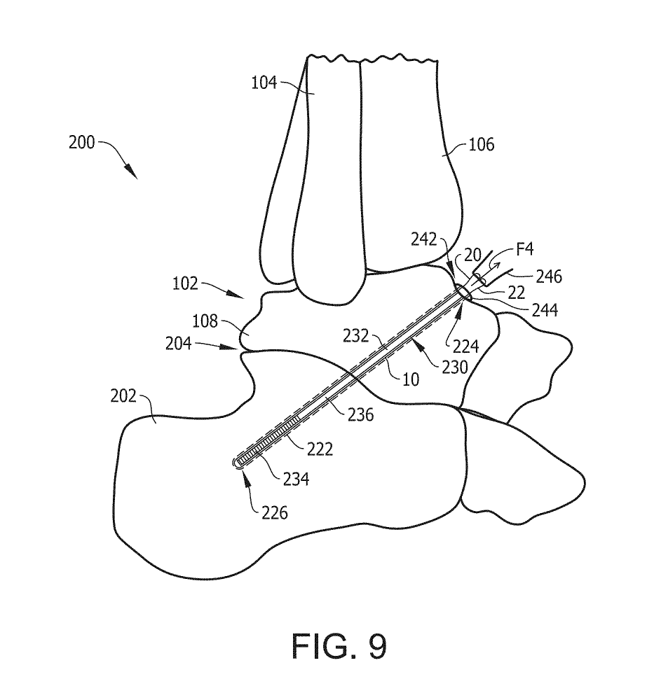

FIGS. 8 and 9 illustrate a second surgical procedure implemented using wire 10 (shown in FIGS. 1 and 2). Although the procedures described herein include using wire 10, it is contemplated that ribbon 50 (shown in FIGS. 3a, 3b, 4a, and 4b) may be used instead of wire 10. As such, the procedures are described below as using wire 10 for convenience only and are not meant to be limiting to only the use of wire 10. More specifically, FIG. 8 is a first view of a region 200 including ankle 102 (shown in FIGS. 8 and 9), on which the second surgical procedure is performed, with a guidewire 220 therethrough in accordance with one aspect of the second surgical procedure, and FIG. 9 is a second view of region 200 with wire 10 disposed therethrough to induce a compressive force across region 200. In the illustrated embodiment, region 200 of FIGS. 8 and 9 depicts an outer side view of a human right ankle 102 including fibula 104, tibia 106, talus 108, and a calcaneus 202. Talus 108 and calcaneus 202 define a subtalar joint 204. In the example embodiment, the second surgical procedure includes subtalar arthrodesis, which may be performed as treatment for subtalar arthritis. To reduce or eliminate the above-described issues with traditional surgical procedures, the subtalar arthrodesis implemented using wire 10 to induce a continuous compressive force across talus 108 and calcaneus 202 is herein described. Directional terms may be understood as follows: with respect to the view of FIGS. 6 and 7, “superior” indicates generally “upwards” in the vertical or longitudinal direction, “inferior” indicates generally “downwards” in the vertical or longitudinal direction, “anterior” indicates generally “right” in the horizontal direction (i.e., towards the front of the patient), “posterior” indicates generally “left” in the horizontal direction (i.e., towards the back of the patient), and “lateral” indicates the “outside” of the patient, or the view of FIGS. 8 and 9.

Although not shown in FIGS. 8 and 9, a number of preparatory steps are performed to access region 200. For example, using a lateral approach to subtalar joint 204 with a first incision, the sinus tarsi 206 is entered, allowing visualization of subtalar joint 204. Care should be taken to avoid the sural nerve, peroneal tendons, and peroneal nerve (not shown). Subtalar joint 204 is distracted, and a pair of join surfaces 208, 210 are prepared by removing any osteocartilagenous surfaces down to healthy bone. Coating surfaces of join surfaces 208, 210 are created to maximize contact area therebetween. A second incision is made over talar neck 212, and talar neck 212 is isolated. Care should be taken to avoid the dorsal neurovascular bundle (not shown).

A first guidewire 220 is drilled across talar neck 212, across subtalar joint 204, and into calcaneus 202. The position of guidewire 220 is verified, for example, fluoroscopically. In one embodiment, a second guidewire (220) is placed parallel to first guidewire 220. A cannulated drill (which may be similar to drill 120, not shown in FIGS. 8 and 9) is passed over the one or more guidewire(s) 220 to form a channel 222 through subtalar joint 204. Channel 222 includes an anterior end 224 and a posterior end 226, which is internal to calcaneus 202. Guidewire(s) 220 and the drill are then removed.

In the illustrated embodiment, as shown in FIG. 9, a fastening device 230 is guided into anterior end 224 of channel 222. Fastening device 230 includes a screw 232, including a threaded portion 234 and a non-threaded portion 236. Wire 10 is coupled to either or both of threaded portion 234 and non-threaded portion 236. Accordingly, as fastening device 230 is inserted into channel 222, wire 10 is guided through channel 222. Threaded portion 234 is secured into calcaneus 202 at posterior end 226 of channel 222, thereby anchoring nitinol wire 10 in calcaneus 202. Free ends 20 and 22 of wire 10 are loose or free on the anterior side of talus 108. In some embodiments, a plate (not shown) is positioned on the anterior side of talus 108, to distribute pressure exerted about anterior end 224 of channel 222.

In the example embodiment, the position of wire 10 in channel 222 is verified (e.g., fluoroscopically), at which point a force F4 is applied axially to wire 10 (i.e., parallel to channel 222). A predetermined amount of force is applied to wire 10, to compress talus 108 and calcaneus 202 together across channel 222. As force F4 is applied to wire 10, fastening device 230 draws talus 108 and calcaneus 202 together. The predetermined amount of force may be determined based on, for example, an amount of compression required between talus 108 and calcaneus 202 to appropriately facilitate healing of subtalar joint 204. In the illustrated embodiment, force F4 applied to ankle 102 is measured using a tensioning device 246. Tensioning device 246 is any device suitable to measure applied force, for example, a spring scale or a force gauge. Using tensioning device 246, force F4 is increased until the measured amount of force F4 is substantially equal to the predetermined desired amount of force.

Once the predetermined amount of force F4 is applied to wire 10 to appropriately compress talus 108 and calcaneus 202 together, free ends 20 and 22 are further coupled to an anchor 242. In the illustrated embodiment, anchor 242 includes a head 244 of fastening device 230. In other embodiments, anchor 242 may include a button, a plate, or any other suitable anchoring device. In the illustrated embodiment, head 244 is positioned to sit flush proximal to talus neck 212. Ends 20 and 22 of wire 10 are anchored, crimped, and/or cut to a suitable length (e.g., approximately flush to the plate, if used, or talus 108). X-ray may be used at this point to verify the position of wire 10. Bone graft may be added, if needed, and the first and second incisions may be closed.

Accordingly, by performing the above-described surgical procedure, the talus 108 and calcaneus 202 are continuously compressed together across channel 222 by wire 10, which continuously exerts an axial restorative or compressive force opposite F4. More specifically, as tension force F4 is applied to wire 10 by tensioning device 246, wire 10 of fastening device 230 stretches to a length longer than its at rest length when no tension force is applied. As described above, wire 10 includes properties that cause wire 10 to contract back to its at rest state (unstretched). As such, when wire 10 is subjected to the constant predetermined desired tension force F3 and then anchored using anchor 244, wire 10 exerts a corresponding constant compressive force f1a (shown in FIG. 2) that continuously compresses talus 108 and calcaneus 202 together for as long as fastening device 230 remains in place.

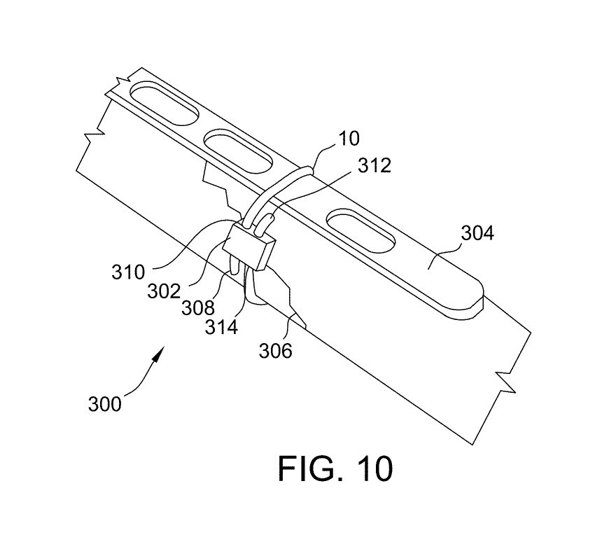

FIG. 10 is a perspective view of a third region 300 of a patient's body on which a third surgical procedure is performed by wrapping a braided superelastic wire 10 around the third region 300. The procedure uses a surgical system including a braided superelastic wire 10, a sleeve 302, and an optional plate 304. Although the procedures described herein include using wire 10, it is contemplated that ribbon 50 (shown in FIGS. 3a, 3b, 4a, and 4b) may be used instead of wire 10. As such, the procedures are described below as using wire 10 for convenience only and are not meant to be limiting to only the use of wire 10.

Plate 304 is coupled to the bone proximate a fracture site 306 and the braided superelastic wire 10 is wrapped around the bone and the plate such that the braided superelastic wire 10 encircles both plate 204 and the bone. Alternatively, the system does not include plate 304 and the braided superelastic wire 10 encircles only the bone proximate the fracture site 306.

In the exemplary embodiment, a first end 308 of the braided superelastic wire 10 is inserted through a first opening 310 in sleeve 302 and a second end 312 of the braided superelastic wire 10 is inserted into a second opening 314 in sleeve 302 adjacent the first opening 310. A tension force is then applied, using a tension device, to at least one of ends 308 and 312 of the braided superelastic wire 10, resulting in a compressive radial force on the bone. The tension force also results in a shift of the braided superelastic wire from an unstretched length in an unstretched, contracted state to a longer stretched length in a stretched state, as described herein. Once a predetermined amount of force is applied to the braided superelastic wire 10, the braided superelastic wire 10 is secured around the bone either by modifying the sleeve 302 to lock the braided superelastic wire 306 within the openings 310 and 314 or by modifying the ends 308 and 312 of the braided superelastic wire 10 to prevent them from slipping out of the sleeve openings 310 and 314. As described herein, the superelastic wire 10 is configured to be biased toward the contracted state when in the stretched state such that the radial compressive force on the bone is maintained while the braided superelastic wire is in the stretched state.

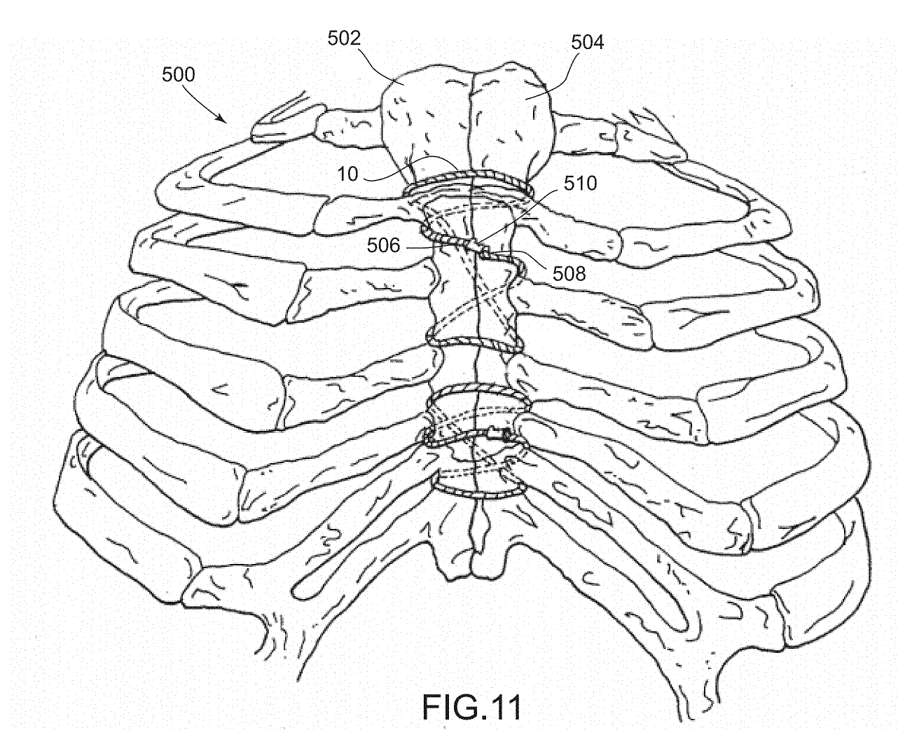

FIG. 11 is a front view of a fourth region 500 of a patient's body on which the third surgical procedure is performed by wrapping braided superelastic wire 10 around the fourth region 500. The procedure is similar to that as described above with respect to eh FIG. 10. Although the procedures described herein include using wire 10, it is contemplated that ribbon 50 (shown in FIGS. 3a, 3b, 4a, and 4b) may be used instead of wire 10. As such, the procedures are described below as using wire 10 for convenience only and are not meant to be limiting to only the use of wire 10.

The surgical procedure begins with wrapping a first bone portion 502 and a second bone portion 504 with braided superelastic wire 10 such that braided superelastic wire 10 forms at least one complete circle around first bone portion 502 and second bone portion 504. In the exemplary embodiment, as shown in FIG. 11, first bone portion 502 includes a first portion of a patient's sternum after the sternum has been split for surgery and second bone portion 504 includes a second portion of the sternum. Generally, first bone portion 502 includes a first portion of any single bone and second bone portion 504 includes a second portion of the bone, and is not limited to the sternum. In an alternative embodiment, first bone portion 502 includes a vertebrae in a patient's neck or back and second bone portion 504 includes at least one adjacent vertebrae in the patient's neck or back. Generally, first bone portion 502 includes any first bone and second bone portion 504 includes any second bone adjacent the first bone and is not limited to vertebrae.

In the exemplary embodiment, after braided superelastic wire 10 is wrapped around bone portions 502 and 504, a tension force is applied, using a tension device as described above, to braided superelastic wire 10, resulting in a compressive radial force on the bone portions 502 and 504. The tension force also results in a shift of the braided superelastic wire from an unstretched length in an unstretched, contracted state to a longer stretched length in a stretched state, as described herein. Once a predetermined amount of force is applied to the braided superelastic wire 10, the braided superelastic wire 10 is secured around the bone portions 502 and 504 to maintain the predetermined amount of tension in the braided superelastic wire 10 and the resulting radial compressive force on the first bone portion 502 and the second bone portion 504. As described herein, the superelastic wire 10 is configured to be biased toward the contracted state when in the stretched state such that the radial compressive force on the bone portions 502 and 504 is maintained while the braided superelastic wire 10 is in the stretched state.

To secure braided superelastic wire 10 in position to maintain the tension force, at least one of a first end 506 and a second end 508 of braided superelastic wire 10 is inserted into a securing mechanism 510, which can then be deformed to secure end 506 and 508. Alternatively, ends 506 and 508 may be twisted together and crimped to secure braided superelastic wire 10. In either securing method, braided superelastic wire 10 may be cut to a desired length before or after securing braided superelastic wire 10.

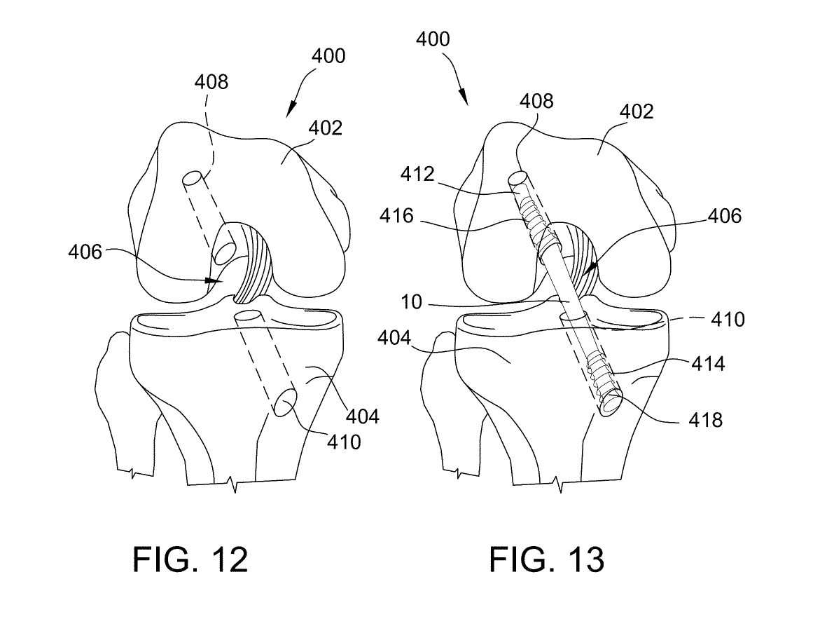

FIG. 12 is a first view of a fifth region 400 of a patient's body on which a fourth surgical procedure is to be performed, and FIG. 13 is a second view of fifth region 400 of a patient's body on which the fourth surgical procedure is to be performed. FIGS. 12 and 13 show and describe using wire 10 or ribbon 50 (shown in FIGS. 3a, 3b, 4a, and 4b) to replace the patient's original tendon or ligament that has torn or ruptured. More specifically, FIGS. 12 and 13 show and describe using wire 10 as a replacement anterior cruciate ligament (ACL) in a patient's knee. However, it is contemplated that wire 10 may be used as a replacement for other tendons and ligaments and is not meant to be limited for use as a replacement ACL. Furthermore, although the procedures described herein include using wire 10, it is contemplated that ribbon 50 may be used instead of wire 10. As such, the procedures are described below as using wire 10 for convenience only and are not meant to be limiting to only the use of wire 10.

Region 400 includes a first bone 402, such as, but not limited to a femur, and a second bone 404, such as, but not limited to a tibia. Femur 402 and tibia 404 are separated by a cavity 406. After removal of the patient's torn ACL, a first opening or tunnel 408 is drilled into femur 402 from outside femur 402 towards cavity 406. Similarly, a second opening or tunnel 410 is drilled into tibia 404 from outside tibia 404 towards cavity 406. As shown in FIG. 13, wire 10 includes a first end 412 and an opposing second end 414. Wire first end is inserted into second tunnel 410 and extends through cavity 406 and into first tunnel 408 such that at least a portion of ends 412 and 414 extend beyond respective tunnels 408 and 410. Alternatively, wire 10 can be inserted into first tunnel 408 and extend into second tunnel 410.

Second end 414 is then coupled to a first anchor 418, which is then secured to tibia 404 to secure wire 10 to tibia 404. In one implementation, as pictured in FIGS. 12 and 13, anchor 418 includes an interference screw positioned within tunnel 410 to secure second end 414 to tibia 404. In another embodiment, anchor 418 is a button structure, such as shown and described in FIG. 7, which is coupled to an exterior surface of tibia 404. In yet another embodiment, anchor 418 includes a screw, which screws second end 414 of wire 10 to the exterior surface of tibia 404 near tunnel 410.

When second end 414 of wire 10 is coupled to tibia 404, a predetermined amount of force is applied axially to wire 10 (i.e., parallel to tunnel 408). First anchor 418 secures second end 414 within tunnel 410, thus preventing wire 10 from pulling out of tibia 404 when tension is applied to wire 10. The predetermined amount of force may be determined based on, for example, a desired amount of yield in wire 10 to appropriately replace the ACL and facilitate healing of region 400 and unrestricted use of the patient's knee. In the illustrated embodiment, the force is applied to first end 412 of wire 10 and is measured using a tensioning device (as shown in FIGS. 7 and 9). The tensioning device is any device suitable to measure applied force, for example, a spring scale or a force gauge. Using the tensioning device, the force is increased until the measured amount of force is substantially equal to a predetermined desired amount of force known to facilitate healing of region 400.

Once the predetermined desired amount of force is applied to wire 10, a second anchor 416 is coupled to first end 412 of wire 10 to secure wire 10 to femur 402. As described above, anchor 416 includes an interference screw positioned within tunnel 408 to secure first end 412 to femur 402. In another embodiment, anchor 416 is a button structure, such as shown and described in FIG. 7, which is coupled to an exterior surface of femur 402. In yet another embodiment, anchor 416 includes a screw, which screws first end 412 of wire 10 to the exterior surface of femur 402 near tunnel 408.

Accordingly, by performing the above-described surgical procedure, the patient's torn ACL is replaced by wire 10, which continuously exerts a constant compressive force to limit movement of femur 402 with respect to tibia 404. More specifically, as the tension force is applied to wire 10, wire 10 stretches to a length longer than its at rest length when no tension is applied. As described above, when wire 10 is secured in such a stretched state, the constant compressive force is maintained because wire 10 includes properties that cause wire 10 to bias towards its contracted or unstretched state when wire 10 is in its stretched state such that a compressive spring force is applied by wire 10 while in the stretched state. In the present example of ACL replacement, the wire 10 is secured between femur 402 and tibia 404 such that wire 10 maintains some yield to enable the patient to bend their knee. As such, when the knee is not bent, the length of wire 10 is shorter than when the patient bends their knee.

In one embodiment, wire 10 or ribbon 50 may also be used to reattach a tendon or ligament to the bone from which it has become separated. Although the procedures described herein include using wire 10, it is contemplated that ribbon 50 (shown in FIGS. 3a, 3b, 4a, and 4b) may be used instead of wire 10. As such, the procedures are described below as using wire 10 for convenience only and are not meant to be limiting to only the use of wire 10. In order to reattach the tendon, an opening is drilled into the bone at the location where the tendon naturally attached to the bone. A first end of the wire is then coupled to an anchor, which is then secured to the bone within the drilled opening such that wire protrudes from the opening. In one implementation, the anchor includes an interference screw positioned within the opening. Alternatively, the anchor can be any anchor that facilitates operation of the wire as described herein.

The opposite end of the wire is then threaded through the tendon at a location on the tendon where the tendon naturally attaches to the bone. In one implementation, the wire is directly coupled to the tendon without another coupling mechanism being used. In another implementation, a suture material is used to couple the tendon to the wire. A second anchor is then coupled to the tendon and to the second end of the wire after the wire passes through the tendon. In one implementation, the second anchor includes a button through which the wire is threaded. The wire may either be directly coupled to the anchor, or suture material may be used to couple the wire to the anchor.

As described above, a predetermined amount of tension force is then applied to the wire to compress the button anchor and the bone. Applying the force causes the button to flatten against the tendon and to pinch tendon against the bone at the natural location from which the tendon has come detached. In the exemplary embodiment of tendon reattachment, a tensioning device, similar to those as described above, is coupled to the wire to measure the amount of force being applied to the wire. Using the tensioning device, the force is increased until the measured amount of force is substantially equal to a predetermined desired amount of force known to facilitate healing of the tendon.

Accordingly, by performing the above-described surgical procedures, the tendon and the bone are continuously compressed together by wire 10, which continuously exerts a constant compressive force opposite. More specifically, as the tension force is applied to the wire by the tensioning device, the wire stretches to a length longer than its at rest length when no tension is applied. As described above, when the wire is secured in such a stretched state, the constant compressive force is maintained because the wire includes properties that cause the wire to bias towards its contracted or unstretched state when the wire is in its stretched state such that a compressive spring force is applied by the wire while in the stretched state. As such, when the wire is subjected to the constant predetermined desired tension force and then anchored, the wire exerts a corresponding constant compressive force f1a (shown in FIG. 2) that continuously compresses the tendon and the bone together for as long as the wire remains in place.

In general, it should be appreciated, that the basic principle illustrated in the above embodiments may be replicated for any two or more components of the body across which continuous compressive force is needed. For example, the above described wire and ribbon may be used to join other two parts of a single bone in the case of a fracture, or used to join two different bones as in the case of a ligament replacement, or used to join a bone to soft tissue as in the case of tendon reattachment. In each case, the principle described herein provides for a longer lasting and more reliable surgical procedure for inducing compressive force across those two components than has been heretofore known.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A surgical procedure for inducing continuous compressive force across two components, said procedure comprising:
inserting a braided superelastic wire through a first end of a channel, the channel extending from a first component to a second component, the braided superelastic wire having an unstretched length in a contracted state;
anchoring the braided superelastic wire at a second end of the channel opposite the first end;
applying a predetermined amount of axial tension to the braided superelastic wire adjacent the second end of the channel, wherein the axial tension stretches the braided superelastic wire to a stretched state having a stretched length that is longer than the unstretched length; and
securing at least one free end of the braided superelastic wire adjacent the second end of the channel to maintain the predetermined amount of axial tension along the braided superelastic wire across the first component and the second component, wherein the braided superelastic wire is configured to be biased toward the contracted state when in the stretched state such that a compressive spring force is applied by the wire while in the stretched state.

2. The surgical procedure of claim 1, wherein applying a predetermined amount of axial tension comprises:
coupling a tensioning device to the braided superelastic wire;
measuring an amount of force applied to the braided superelastic wire; and
changing the axial tension until the measured amount of force is substantially equal to the predetermined amount of axial tension.

3. The surgical procedure of claim 1, further comprising drilling the channel across the first and second components using a cannulated drill.

4. The surgical procedure of claim 1, wherein anchoring the braided superelastic wire at the second end of the channel comprises:
threading the wire through at least one button aperture of a button; and
positioning the button adjacent the second end of the channel.

5. The surgical procedure of claim 1, wherein anchoring the braided superelastic wire at the second end of the channel comprises:
coupling the braided superelastic wire to a fastening device including a threaded portion; and
securing the threaded portion of the fastening device adjacent the second end of the channel.

6. The surgical procedure of claim 1, further comprising fluoroscopically verifying a position of the braided superelastic wire.

7. The surgical procedure of claim 1, further comprising applying, using the braided superelastic wire, a constant amount of compressive force across the first component and the second component after the braided superelastic wire is secured proximate the second end.

8. A surgical system comprising:
a braided superelastic wire configured to induce a compressive force across a first component and a second component through a channel having a first end through at least a portion of the first component and a second end through at least a portion of the second component, when an axial force is applied thereto, the braided superelastic wire having an unstretched length and further configured to stretch to a stretched length when the axial force is applied, wherein the stretched length is longer than the unstretched length, said braided superelastic wire further configured to contract back to its unstretched length when the axial force is removed; and
an anchor configured to anchor said braided superelastic wire adjacent the second end of the channel and to prevent said braided superelastic wire from being displaced through said channel upon application of the predetermined amount of axial force to said braided superelastic wire.

9. The surgical system of claim 8, further comprising a cannulated drill configured to drill the channel across the first and second components.

10. The surgical system of claim 8, wherein said anchor comprises a button comprising at least one button aperture, said braided superelastic wire configured to be threaded through said at least one button aperture to secure said braided superelastic wire to said button.

11. The surgical system of claim 8, wherein said anchor comprises a threaded portion, said braided superelastic wire configured to be coupled to said threaded portion, and said threaded portion configured to be secured to the second component adjacent the second end of the channel.

12. The surgical system of claim 8, further comprising a tensioning device configured to increase the axial force until the axial force is substantially equal to a predetermined amount of force.

13. A surgical procedure for inducing continuous compressive force to stabilize a bone having a fracture, said procedure comprising:
encircling the bone with a braided superelastic wire proximate the fracture, the braided superelastic wire having an unstretched length in a contracted state;
inserting a first end of the braided superelastic wire through a first opening formed in a sleeve;
inserting a second end of the braided superelastic wire through a second opening formed in the sleeve;
applying a predetermined amount of tension to at least one of the first and second ends of the braided superelastic wire such that a radial compressive force acts on the bone, wherein the tension stretches the braided superelastic wire to a stretched state having a stretched length that is longer than the unstretched length; and
securing the braided superelastic wire around the bone to maintain the predetermined amount of tension in the braided superelastic wire and the resulting radial compressive force on the bone, wherein the braided superelastic wire is configured to be biased toward the contracted state when in the stretched state such that the radial compressive force is applied by the braided superelastic wire while in the stretched state.

14. The surgical procedure of claim 13, wherein applying a predetermined amount of tension comprises:
coupling a tensioning device to the braided superelastic wire;
measuring an amount of force applied to the braided superelastic wire; and
changing the measured amount of force until the measured amount of force is substantially equal to the predetermined amount of tension.

15. The surgical procedure of claim 13, wherein securing the braided superelastic wire around the bone comprises deforming the sleeve to lock the braided superelastic wire within the first and second openings.

16. The surgical procedure of claim 13, wherein securing the superelastic wire around the bone comprises modifying the first end and the second end of the braided superelastic wire to prevent the braided superelastic wire from sliding back through the first and second openings.

17. The surgical procedure of claim 13, further comprising cutting the braided superelastic wire to a desired length after applying the predetermined amount of tension.

18. The surgical procedure of claim 13, further comprising coupling a plate to the bone, wherein encircling the bone with a braided superelastic wire comprises encircling the bone and the plate with the braided superelastic wire.

19. A surgical procedure for inducing continuous compressive force between hard tissue and soft tissue, said procedure comprising:
anchoring a first end of a braided superelastic wire within a hole formed in the hard tissue at a location where the soft tissue is to be attached;
coupling a second end of the braided superelastic wire to the soft tissue at a location on the soft tissue where the soft tissue attaches to the hard tissue, the braided superelastic wire having an unstretched length in a contracted state;
applying a predetermined amount of tension to the second end of the braided superelastic wire, wherein the tension stretches the braided superelastic wire to a stretched state having a stretched length that is longer than the unstretched length; and securing the second end of the braided superelastic wire to the soft tissue to maintain the predetermined amount of tension in the braided superelastic wire between the hard tissue and the soft tissue, wherein the braided superelastic wire is configured to be biased toward the contracted state when in the stretched state such that a (compressive) spring force is applied by the braided superelastic wire while in the stretched state.

20. The surgical procedure of claim 19, wherein anchoring the braided superelastic wire within a hole formed in the hard tissue comprises:
drilling a hole in the hard tissue′
coupling the braided superelastic wire to an anchor
coupling the anchor to the hard tissue within the hole such that the braided superelastic wire protrudes form the hole.

21. The surgical procedure of claim 19, wherein applying a predetermined amount of tension comprises:
coupling a tensioning device to the braided superelastic wire;
measuring an amount of force applied to the braided superelastic wire; and changing the measured amount of force until the measured amount of force is substantially equal to the predetermined amount of tension.

22. The surgical procedure of claim 19, wherein coupling a second end of the braided superelastic wire to the soft tissue comprises coupling the second end of the braided superelastic wire directly to the soft tissue.

23. The surgical procedure of claim 19, wherein coupling a second end of the braided superelastic wire to the soft tissue comprises coupling a suture material between the second end of the braided superelastic wire and the soft tissue.

24. The surgical procedure of claim 19 further comprising coupling an anchor to the soft tissue such that the soft tissue is positioned between the hard tissue and the anchor.

25. The surgical procedure of claim 24, wherein securing the second end of the braided superelastic wire to the soft tissue comprises coupling the second end of the braided superelastic wire directly to the anchor.

26. The surgical procedure of claim 24, wherein securing the second end of the braided superelastic wire to the soft tissue comprises coupling a suture material between the second end of the braided superelastic wire and the anchor.

27. A surgical procedure for inducing continuous compressive force between a first hard tissue member and a second hard tissue component, said procedure comprising:
anchoring a first end of a braided superelastic wire within a hole formed in the first hard tissue component, the braided superelastic wire having an unstretched length in a contracted state;
applying a predetermined amount of tension to the second end of the braided superelastic wire, wherein the tension stretches the braided superelastic wire to a stretched state having a stretched length that is longer than the unstretched length; and
anchoring a second end of the braided superelastic wire within a hole formed in the second hard tissue component to maintain the predetermined amount of tension in the braided superelastic wire between the first hard tissue component and the second hard tissue component, wherein the braided superelastic wire is configured to be biased toward the contracted state when in the stretched state such that a (compressive) spring force is applied by the braided superelastic wire while in the stretched state.

28. A surgical procedure for inducing continuous compressive force between a first bone portion and a second bone portion, said procedure comprising:
wrapping the first bone portion and the second bone portion with a braided superelastic wire such that the braided superelastic wire forms at least 1 complete circle around the first bone portion and the second bone portion, the braided superelastic wire having an unstretched length in a contracted state;
applying a predetermined amount of tension to the braided superelastic wire such that a radial compressive force acts on the first bone portion and the second bone portion, wherein the tension stretches the braided superelastic wire to a stretched state having a stretched length that is longer than the unstretched length; and
securing the braided superelastic wire around the first bone portion and the second bone portion to maintain the predetermined amount of tension in the braided superelastic wire and the resulting radial compressive force on the first bone portion and the second bone portion, wherein the braided superelastic wire is configured to be biased toward the contracted state when in the stretched state such that the radial compressive force is applied by the braided superelastic wire while in the stretched state.

29. The surgical procedure of claim 28, wherein applying a predetermined amount of tension comprises:
coupling a tensioning device to the braided superelastic wire;
measuring an amount of force applied to the braided superelastic wire; and
changing the measured amount of force until the measured amount of force is substantially equal to the predetermined amount of tension.

30. The surgical procedure of claim 28, wherein wrapping the first bone portion and the second bone portion with a braided superelastic wire comprises wrapping a first vertebrae and a second vertebrae with a braided superelastic wire.

31. The surgical procedure of claim 28, wherein wrapping the first bone portion and the second bone portion with a braided superelastic wire comprises wrapping a first portion of a sternum and a second portion of a sternum with a braided superelastic wire.

32. The surgical procedure of claim 28, further comprising cutting the braided superelastic wire to a desired length after applying the predetermined amount of tension.

33. The surgical procedure of claim 28, wherein wrapping the first bone portion and the second bone portion with a braided superelastic wire comprises wrapping a first bone and a second bone with a braided superelastic wire.

34. The surgical procedure of claim 28, wherein wrapping the first bone portion and the second bone portion with a braided superelastic wire comprises wrapping a first portion of a bone and a second portion of the bone with a braided superelastic wire.

35. The surgical procedure of claim 28, wherein securing the braided superelastic wire around the first bone portion and the second bone portion comprises twisting opposing ends of the braided superelastic wire together.

36. The surgical procedure of claim 28, wherein securing the braided superelastic wire around the first bone portion and the second bone portion comprises inserting at least one of opposing ends of the braided superelastic wire into a securing mechanism.
